(19) United States
(12) Patent Application Publication
(10) Pub. No.: US 2008/0268524 A1
(43) Pub. Date: Oct. 30, 2008

(54) Method of Modifying the Viability of a Lyophilized Microorganism by Treating the Growth Medium Thereof with Gases

(76) Inventors: Remy Cachon, Dijon (FR); Carole Delbeau, Cesy (FR); Gilles Feron, Pontailler-Sur-Saone (FR); Henry Ledon, Versailles (FR)

Correspondence Address: AIR LIQUIDE;Intellectual Property, 2700 POST OAK BOULEVARD, SUITE 1800, HOUSTON, TX, 77056, US

(21) Appl. No.: 11/816,628

(22) PCT Filed: Feb. 16, 2006
(86) PCT No.: PCT/FR06/50140

§ 371 Date: Jul. 17, 2008

(30) Foreign Application Priority Data
Feb. 22, 2005 (FR) 0550483

(51) Int. Cl. C12N 1/04

(52) U.S. Cl. 435/260

(57) ABSTRACT

The invention relates to a method of producing lyophilized microorganisms, such as lyophilized bacteria, of the type in which a culture medium is inoculated with one or more strains of microorganisms during one of the steps of the method. The invention is characterised in that, prior to the inoculation step, the culture medium is treated with a treatment gas comprising an inert gas or a reducing gas or a mixture of such inert and reducing gases, in order to obtain a determined redox potential value Eh for the medium, which is less than the value obtained when the medium is in equilibrium with the air.

The present invention relates to the field of the production of freeze-dried microorganisms, in particular freeze-dried bacteria, and it focuses in particular on freeze-dried lactic acid bacteria, by endeavoring to propose novel operating conditions for increasing the viability of freeze-dried microorganisms during their subsequent storage.

It should be recalled that all types of microorganisms (bacteria, yeasts, molds, etc) can, a priori, be freeze-dried; freeze-drying is used in particular for conserving strains in microorganism collections. Freeze-drying is, for example, carried out on lactic acid bacteria, probiotic strains and yeasts, and for very diverse industrial uses.

It should also be recalled that microorganisms are in particular used for the bioconversion of starting materials in the case of:
the manufacture of a finished product (cheese, yoghurt, wine, beer, bread, etc),
the manufacture of a biomass intended for human or animal nutrition (extract and powder of yeast, of probiotics, etc),
the production of specific molecules of interest (enzymes, antibiotics, amino acids, flavorings, etc),
the purification of industrial effluents, treatment of organic waste, etc, etc.

Considering, in the subsequent text, the example of lactic acid bacteria, the industrial exploitation of lactic acid bacteria as leavens and probiotic cultures is highly dependent on the preservation technologies used in order to guarantee stable cultures, i.e. cultures which are viable and active in the long term.

Freezing and freeze-drying are commonly used for this purpose, but these techniques introduce unwanted side effects, which are protein denaturation and reduced cell viability. Studies carried out in Lactobacillus bulgaricus (Lb. bulgaricus) during drying and storage have identified factors, such as the temperature and the water activity of the dried powders, as essential parameters which could affect survival (see, in particular, the work by Castro et al., published in 1995 in Applied Microbiology and Biotechnology. 44:172-176). The loss of viability of the powders is the result of cell damage; the preferential targets are the cell wall, the cell membrane and the DNA, and also oxidation of the lipid membranes (see the article by Carvelho et al., published in 2004, in International Dairy Journal. 14:835-847).

Optimization of the survival of frozen or freeze-dried lactic acid bacterial cultures, and the storage thereof for long periods, are therefore of definite importance, both technological and economic importance.

It should be recalled that the production of freeze-dried strains is carried out from a parent strain and in general comprises 4 steps: inoculation, culturing in a tank, concentration, storage. Reference will be made, for example, to the following works: “Freeze Drying and Advanced Food Technology”, Goldblith et al., Academic Press in 1975, or “Traité de Lyophilisation” [Dissertation on freeze-drying], Louis Rey, published by Hermann in 1960.

The inoculation is carried out using a culture which is concentrated and in an optimal physiological state. Several techniques can be used, among which are:
the use of a tank starter,
the method by successive subculturings,
direct inoculation,
continuous inoculation.

The step of culturing per se is carried out in a tank, with or without shaking, in a culture medium whose composition is suitable for the specific needs of each microorganism. As will be seen, for example, in the works cited above, the composition of the culture medium can be extremely varied, but mention is commonly made of the presence of one or more elements from polysaccharides, glycerol, milk, glucose, etc.

Similarly, the parameters such as, for example, the pH, the temperature or the dissolved oxygen pressure can be regulated.

Various types of culture exist:
discontinuous or batch culture, used in particular for the production of lactic ferments or of bread-making yeast,
semicontinuous or “fed-batch” culture, used for example for the production of ferments sensitive to a product of the fermentation or for the production of a biomass sensitive to inhibition by the fermentation substrate,
continuous culture with or without recycling, the latter being used in particular for the production of ferments or of molecules of interest, and for the biological purification of wastewater.

The conservation step can be carried out in liquid form, by freezing, by cryoconservation, by freeze-drying or by drying. Protective agents are used in order to preserve the microorganisms against the harmful effects of the conservation treatments.

Freeze-drying is, as is known, a low-temperature dehydration operation which consists in removing, by sublimation, the majority of the water contained in the product after freezing.

It is known, moreover, that the redox potential (often referred to in the literature as Eh) is a physiochemical parameter which, by virtue of its nature, is present in all media provided that the latter contain at least one molecule which can change from an oxidized state to a reduced state, and vice versa. For this reason, its effect can be seen on all cell functions. Its action has been shown on various types of microorganisms, and in particular of bacterial strains:
the addition of chemical reducing agents to culture media has already made it possible to significantly modify the growth and the metabolic fluxes in Corynebacterium glutamicum, Clostridium acetobutylicum, Sporidiobolus, and Escherichia coli.
a reducing Eh which is fixed via gases has made it possible to modify the metabolic fluxes in Saccharomyces cerevisiae with an increase in the glycerol/ethanol ratio and the accumulation of storage sugars with an increase in yeast survival during conservation in the liquid state (reference will be made to document FR-2 811 331 in the applicant's name).

In the industrial medium, the Eh is already indirectly taken into account through oxygen, the inhibitory effect of which on lactic acid bacteria has been clearly identified. This effect is due to their inability to synthesize cytochromes and enzymes with a heme nucleus.

It is also possible, by acting on the Eh, to modify the survival of probiotic ferments, metabolic fluxes, and the production and/or the stability of flavoring molecules. All these results have been obtained following a modification of the Eh by the microorganisms themselves, by oxidoreductive molecules or by thermal treatment.

The survival of microorganisms after freeze-drying and during conservation is dependent on many factors, including the initial concentration of microorganisms, the growth conditions, the growth medium, the drying medium and the rehydration conditions.

In conservation methods of freezing or freeze-drying type, the growth medium is therefore an important parameter to be controlled; each of the constituents of the medium can provide protection, in particular by allowing the accumulation of solutes, the production of exopolysaccharides and the modification of the membrane lipid profile, in particular by increasing the unsaturated fatty acid/saturated fatty acid ratio.

Previous studies have shown that cell damage appeared during freeze-drying methods and that antioxidants added to the freeze-drying medium made it possible to protect the membrane lipids against this damage, and have shown the advantage of adding, to a concentrate of lactic acid bacteria (after culturing, i.e. to the freeze-drying or freezing medium), chemical antioxidant molecules in order to protect the membrane lipids against the oxidation can occur during freeze-drying or during storage (reference will be made, for example, to document WO 03/018778 or else to the studies by Fonseca et al., published in 2003 in International Dairy Journal. 13:917-926).

It is consequently seen that there exists a real need to be able to provide a novel method of producing freeze-dried microorganisms, and in particular freeze-dried bacteria, which makes it possible to improve the viability of a strain with respect to freeze-drying.

In addition, with a nutritional pharmaceutical or veterinary application in mind, the variation in the Eh should involve compounds which do not modify the characteristics of the product and which preserve the innocuousness of the products.

As will be seen in greater detail in the subsequent text, it is proposed, according to the present invention, to modify the redox potential of the culture medium of the strain using a treatment gas comprising an inert gas and/or a reducing gas, before inoculation.

According to the invention, the culture medium is therefore treated with a treatment gas comprising an inert gas such as nitrogen, argon, helium or carbon dioxide or a mixture of inert gases, or a reducing gas such as hydrogen, or a mixture of such inert and reducing gases, in order to obtain a redox potential value Eh which is less than the value obtained when the mixture is in equilibrium with the air, before the step of inoculation of the medium.

The treatment, i.e. the gas/liquid contact, can be carried out according to one of the methods well known, moreover, to those skilled in the art, such as bubbling through the medium using a sintered glass funnel, membrane or a porous substance, agitation by means of a hollow-shafted turbine, use of a hydroinjector, etc.

The present invention therefore relates to a method of producing freeze-dried microorganisms, in particular freeze-dried bacteria, in particular freeze-dried lactic acid bacteria, of the type in which, during one of the steps of the production method, the culture medium is inoculated with one or more strains of microorganisms, and characterized in that, before the inoculation step, the culture medium is treated with a treatment gas comprising an inert gas or a reducing gas or a mixture of such gases, in order to obtain a given redox potential value Eh for the medium which is less than the value obtained when the medium is in equilibrium with the air.

In preferred embodiments of the invention, use may optionally be made, in addition, of one and/or the other of the following arrangements:
said desired redox potential value is at least 100 mV less than the value obtained when the medium is in equilibrium with the air;
said desired redox potential value is negative;
the inoculation is carried out indirectly by virtue of the fact that a preculture is carried out beforehand, which preculture is subsequently used for said inoculation, and that, before inoculation of the preculture, the preculture medium is treated with a pretreatment gas comprising an inert gas or a reducing gas or a mixture of such gases, in order to obtain a given redox potential value Eh for the preculture medium which is less than the value obtained when the preculture medium is in equilibrium with the air;
said treatment or pretreatment gas is hydrogen or comprises hydrogen;
said treatment or pretreatment gas is nitrogen or comprises nitrogen;
said treatment or pretreatment gas is a mixture of hydrogen and nitrogen;
said treatment or pretreatment gas is argon or comprises argon;
said treatment or pretreatment gas comprises hydrogen and/or nitrogen and an additional gas which is acceptable from the point of view of the subsequent use of the freeze-dried microorganisms thus produced;
said treatment or pretreatment gas also comprises an additional gas which is chosen from inert gases, in particular helium, and from oxygen, carbon dioxide and nitrous oxide and mixtures thereof in any proportions, preferably from carbon dioxide and oxygen and mixtures thereof;
said treatment or pretreatment gas is a mixture of hydrogen and carbon dioxide.

In addition, as will also be seen in detail below, the freeze-dried microorganisms thus obtained have improved properties, in particular in terms of resistance of the strain to freeze-drying and resistance during the subsequent conservation of said strain.

Other characteristics and advantages of the invention will emerge from the detailed examples below which relate to the field of lactic acid bacteria.

Sterile skimmed milk (4.5 liters) was treated for 1 hour in a modified 5-liter Schott flask by bubbling through two different gases at a flow rate of 150 ml/min; a control condition without bubbling was also carried out:
control (reference);
nitrogen (according to the invention);
nitrogen/hydrogen mixture, 96/4 by volume (according to the invention).

Three tests were carried out for each condition.

The redox potential values thus attained, related back to pH 7 (by formulae well known to those skilled in the art, such as the Leistner and Mirna equation, which makes it possible to relate the Eh of a medium of pH=x back to its value at pH 7), according to the gas used, measured with a Mettler Toledo probe, are as follows:

| Control | Nitrogen | Nitrogen/hydrogen |
|---|---|---|
| +300 mV | +200 mV | −305 mV |

The media (sterile skimmed milk) were then inoculated with a probiotic Lactobacillus casei strain and then placed in an incubator at 37° C. for 72 hours.

After 72 hours of growth, the cultures are recovered and freeze-drying fillers (of conventional type, as mentioned above in the present description) are added. The mixture is neutralized using a solution of calcium hydroxide. The preparation thus obtained is placed in trays in order to undergo freeze-drying.

The bacteria in the culture medium are therefore counted, after 72 hours of growth, and the freeze-drying fillers are added. The results obtained are given in table 1 below.

The statistical treatment is carried out on the counts obtained for the 3 treatments. “ns” indicates that the differences observed are not significant (Newman-Keuls test at 5%).

It is concluded therefrom that there is no significant difference in production of biomass between the two treatment gas mixtures studied.

TABLE 1
Biomass of Lb. casei expressed as CFU/ml and obtained in a liquid
medium after 72 hours of growth and addition of fillers under the
3 treatments of the culture medium (mean of 3 tests in each case):

| | | Percentage of biomass |
|---|---|---|
| Treatment of | | acquired relative to |
| the culture | Biomass | the control |
| medium | CFU/ml | % |
| Control | 6.67 · 10⁸ (ns) | |
| N₂ | 8.33 · 10⁸ (ns) | +25% |
| N₂/H₂ | 8.67 · 10⁸ (ns) | +30% |

The count obtained on the freeze-dried powder at D+1, that is to say conserved for 1 day after it has been obtained, i.e. the counts are carried out on the day which followed the obtaining of the powder, will now be examined below.

These results are given in table 2 below.

A statistical treatment was carried out on the counts obtained for the 3 treatments. It shows that the differences observed between, firstly, the N₂/H₂ treatment and, secondly, the N₂ and control treatments are significant (Newman-Keuls test at 5%).

It is therefore observed that the count for the powders obtained from the cultures carried out on a medium treated with nitrogen-hydrogen is greater than the count relating to those carried out on control or N₂-treated medium. It is noted that the results obtained with nitrogen already show a substantial increase relative to the control.

An increase in the resistance of the strains with respect to the freeze-drying is therefore observed for the cells cultured with N₂/H₂ treatment, demonstrating a positive effect of the treatment of the culture medium on the resistance of the strain with respect to freeze-drying.

TABLE 2
Biomass of Lb. casei expressed as CFU/g of powder
as a function of the initial treatment of the growth
medium after 1 day (mean of 3 tests per case)

| | | Percentage of biomass |
|---|---|---|
| Treatment of | | acquired relative to |
| the culture | Biomass | the control |
| medium | CFU/g of powder | % |
| Control | 6.88 · 10⁸ | |
| N₂ | 8.41 · 10⁸ | +25% |
| N₂/H₂ | 1.04 · 10⁹ | +52% |

The results of counts obtained on the powder at D+60 (i.e. conserved for 60 days after it has been obtained) will now be examined in the subsequent text. They are given in table 3 below.

TABLE 3
Biomass of Lb. casei expressed as CFU/g of powder as a function
of the initial treatment of the growth medium, after 60 days of
conservation at ambient temperature (mean of 3 tests per case)

| | | Percentage of biomass |
|---|---|---|
| Treatment of | | acquired relative to |
| the culture | Biomass | the control |
| medium | CFU/g of powder | % |
| Control | 1.24 · 10⁷ | |
| N₂ | 2.17 · 10⁷ | +75% |
| N₂/H₂ | 4.02 · 10⁷ | +223% |

Here again, a statistical treatment was carried out on the counts obtained for the 3 treatments. It shows significant differences between the three different treatments (Newman-Keuls test at 5%).

These results indicate a count which is higher for the cells cultured on medium treated with N₂/H₂ compared with those cultured with N₂ treatment, for which the count is also significantly higher than that obtained with the control.

It may clearly be concluded that the positive effect of the initial treatment of the growth medium of the strain with N₂ and N₂/H₂, on its resistance with respect to freeze-drying, is confirmed and even amplified during conservation. This shows the advantage of using the gases for modifying the Eh of the growth medium before the inoculation step per se, on the resistance of the strain with respect to freeze-drying and on its resistance during its conservation.

1-11. (canceled)

12. A method of producing freeze-dried microorganisms, in particular freeze-dried bacteria, which method is of the type in which, during one of the steps of the production method, a culture medium is inoculated with one or more strains of microorganisms, and is characterized in that, before the inoculation step, the culture medium is treated with a treatment gas comprising an inert gas or a reducing gas or a mixture of such gases, in order to obtain a given redox potential value Eh for the medium which is less than the value obtained when the medium is in equilibrium with the air.

13. The method of production of claim 12, wherein said desired redox potential value is at least 100 mV less than the value obtained when the medium is in equilibrium with the air.

14. The method of production of claim 13, wherein said desired redox potential value is negative.

15. The method of production of claim 12, wherein the inoculation is carried out indirectly by virtue of the fact that a preculture is carried out beforehand, which preculture is subsequently used for said inoculation, and in that, before inoculation of the preculture, the preculture medium is treated with a pretreatment gas comprising an inert gas or a reducing gas or a mixture of such gases, in order to obtain a given redox potential value Eh for the preculture medium which is less than the value obtained when the preculture medium is in equilibrium with the air.

16. The method of production of claim 12, wherein said treatment or pretreatment gas is hydrogen or comprises hydrogen.

17. The method of production of claim 12, wherein said treatment or pretreatment gas is nitrogen or comprises nitrogen.

18. The method of production of claim 12, wherein said treatment or pretreatment gas is a mixture of hydrogen and nitrogen.

19. The method of production of claim 12, wherein said treatment or pretreatment gas is argon or comprises argon.

20. The method of production of claim 12, wherein said treatment or pretreatment gas comprises hydrogen and/or nitrogen and an additional gas which is acceptable from the point of view of the subsequent use of the freeze-dried microorganisms thus produced.

21. The method of production of claim 12, wherein the treatment or pretreatment gas also comprises an additional gas which is chosen from inert gases, in particular helium, and from oxygen, carbon dioxide and nitrous oxide and mixtures thereof in any proportions, preferably from carbon dioxide and oxygen and also mixtures thereof.

22. The method of production of claim 12, wherein said treatment or pretreatment gas is a mixture of hydrogen and carbon dioxide.
